(19) United States
(12) Patent Application Publication
(10) Pub. No.: US 2013/0236980 A1
(43) Pub. Date: Sep. 12, 2013

(54) Methods, Devices, Systems and Compositions for Detecting Gases

(71) Applicants: Eugene W. Moretti, Durham, NC (US); Robert Lavin Wood, Cary, NC (US); Allan Bruce Shang, Wake Forest, NC (US)

(72) Inventors: Eugene W. Moretti, Durham, NC (US); Robert Lavin Wood, Cary, NC (US); Allan Bruce Shang, Wake Forest, NC (US)

(73) Assignee: Respirion, LLC

(21) Appl. No.: 13/609,024

(22) Filed: Sep. 10, 2012

Related U.S. Application Data
(60) Provisional application No. 61/609,603, filed on Mar. 12, 2012.

(51) Int. Cl. G01N 21/78; A61B 5/08; G01J 1/50

(52) U.S. Cl. 436/133; 422/86; 600/532

(57) ABSTRACT

A method of monitoring a respiratory stream can be provided by monitoring color change of a color change material to determine a CO2 level of the respiratory stream in contact with the color change material by emitting visible light onto the color change material. Related devices, systems, and compositions are also disclosed.

### CROSS-REFERENCE TO RELATED APPLICATION

This Application claims priority to U.S. Provisional Patent Application No. 61/609,603, entitled Methods and Apparatus for Detecting Carbon Dioxide Levels, filed on Mar. 12, 2012, the disclosure of which is entirely incorporated herein by reference.

### FIELD OF INVENTION

The present invention relates to the measurement of gas levels, and more specifically, to measuring respiratory gases.

### BACKGROUND

First responders, respiratory therapists and critical care personnel perform emergency laryngoscopy and intubation under a variety of conditions and under great duress. Securing a patent, viable and protected airway is one of the paramount steps of a successful resuscitation. Often times airway manipulation and instrumentation are performed in suboptimal conditions by inexperienced or lightly trained personnel. These procedures have the potential for disaster if they result in an esophageal intubation, causing hypoxia, anoxia, and cardiopulmonary arrest if allowed to continue unrecognized.

Capnography, the measurement of CO₂ in expired or respirated gases has been commonly used in the operating room setting for several years. Capnography readily identifies situations that can lead to hypoxia if left undetected and dealt with. For example, one use of a CO₂ measuring device is to confirm proper endotracheal tube placement during general anesthesia. By identifying improper placement, the provider can then rectify potential hypoxic conditions before hypoxia can actually lead to severe brain damage. Recently the use of capnography has been extended outside of the operating room arena to include emergency rooms, intensive care units, endoscopic suites, radiographic suites and first responders at catastrophic events (e.g. motor vehicle or industrial accidents).

The current standard of care for collect endotracheal tube placement calls for multiple methods of confirmation, one of which could be a carbon dioxide detector. Typically, however, the gold standard method used to confirm proper placement is a capnographic waveform monitor. Unfortunately, this monitor may be a complex electronic device only capable of functioning in highly controlled environments, such as an operating room. In many cases, these devices are not available, suited, or adapted for the location in which these procedures may be necessary.

Other types of endotrachael tube placement confirmation may be a disposable colorimetric detector. This type of detector confirms the presence of CO₂ via a visible color change in equipment or test strip when exposed to exhaled gases containing CO₂. This device detects CO₂ via a chemical reaction which causes a color shift in a reagent containing substrate contained within the device. This device has limitations which may include lack of quantifiable results, relative insensitivity, time dependent and temperature sensitive decay of reagents, and poor visibility in less than optimal light conditions.

Colorimetric detectors are generally useful as qualitative indicators of the presence or absence of CO₂. Various methods have been disclosed for quantitative detection of CO₂ in respired gas samples. However limitations of these devices may be that they may not provide useful feedback during various patient procedures such as cardiopulmonary resuscitation and/or ventilation. These simple detectors may not add value to patient outcomes beyond informing a simple gate decision of whether CO₂ is present or absent in respiratory gases.

CO₂ concentration at the end of a breath can represent the end tidal carbon dioxide concentration (PETCO₂). Decreases in cardiac output and pulmonary blood flow can result in decreases in PETCO₂. Correspondingly, increases in cardiac output and pulmonary blood flow result in better perfusion of the alveoli and a rise in PETCO₂. The relationship between cardiac output and PETCO₂ has been determined to be logarithmic. Therefore capnography can detect the presence of pulmonary blood flow even in the absence of major pulses, and it can indicate changes in pulmonary blood flow caused by alterations in cardiac rhythm. Initial data samples reveal that the PETCO₂ may correlate with coronary perfusion pressure. This correlation between perfusion pressure and PETCO₂ is likely to be secondary to the relationship between PETCO₂ and cardiac output.

Capnographic measurements have been evaluated to predict outcomes in cardiac arrest. A study involving 127 patients revealed that only one patient with a PETCO₂ less than 10 mm Hg during resuscitation survived to hospital discharge. In another prospective investigation involving 139 adult victims of out-of-hospital, non-traumatic cardiac arrest, no patient with an average PETCO₂ less than 10 mm Hg upon initial resuscitation survived. The analysis of these studies concluded that PETCO₂ can be correlated with resuscitation and outcome in cardiopulmonary resuscitation (CPR). Moreover, another application of capnography in this setting is to provide feedback to optimize chest compressions during CPR. Monitoring PETCO₂ may detect inadequate chest compressions secondary to fatigue that could result in a sub-optimal cardiac output.

Capnography is gaining increasing acceptance during the resuscitation of trauma victims. PETCO₂ is a marker of traumatic physiology, as it reflects changes in cardiac output. Recently a study involving 191 blunt trauma patients revealed that PETCO₂ may be of value in predicting outcome from major trauma. In this investigation only 5% of patients with a PETCO₂ less than 10 mm Hg survived to hospital discharge. Other studies have shown capnography to be of value in providing optimum ventilation in pre-hospital major trauma victims. Patients monitored using capnography had a statistically significant higher incidence of normoventilation (normal CO₂ levels in the blood) compared to those who were not managed with capnography (63.2% vs. 20% p<0.0001).

Some previous CO₂ detectors make use of an electrochemical detection device referred to collectively as “chemiresistors”. Such devices respond to the absorption of target chemical species by undergoing a change in ohmic resistance. In many chemiresistor designs, the change in ohmic resistance may provide a quantitative basis for measurement of the absorbed species. Chemiresistors may generally be comprised of an electrically insulating substrate, with at least one surface having two or more conductive electrode layers spaced apart thereon. These electrodes may comprise a metallic layer, and they may have an interdigitated geometric form. A chemiresistive layer or “ink” may cover two or more electrode layers, and act as the “absorber” that attracts the analyte species of interest. Voltage applied to the electrodes will induce a current flow within the chemiresistive ink layer. Measurement of this current may provide a quantitative basis for detection of absorbed analyte.

Absorption of a species by a chemiresistive layer results in changes in the layer's physical and/or chemical properties, resulting in a change in ohmic resistance. For example, a chemiresistive ink may comprise finely divided carbon particles in a polymeric binder. The proportion of binder and particles may be chosen such that the layer has a first ohmic resistance. Upon absorption of an organic compound having affinity for the polymeric binder, the layer may undergo swelling which causes the particles to generally move out of contact, resulting in high ohmic resistance. The change in ohmic resistance due to swelling may be in proportion to the organic compound. Heating of the layer may desorb the organic compound, regenerating the layer for a new cycle of measurement.

There are several limitations that currently exist with the prior art. None of the prior art addresses the need for a gaseous CO₂ sensor that can be battery powered, portable, with fast response time, immune to humidity and condensation, that provides quantitative measurement. The inventive aspects of the present invention address these limitations.

### SUMMARY

Embodiments according to the invention can provide methods, devices, systems, and compositions for monitoring gases. Pursuant to these embodiments, a method of monitoring a respiratory stream can be provided by monitoring color change of a color change material to determine a CO2 level of the respiratory stream in contact with the color change material by emitting visible light onto the color change material.

In some embodiments according to the invention, the method can further include sensing the color change using a sensor to detect a portion of the emitted visible light reflected from the color change material. In some embodiments according to the invention, the method can further include determining the CO2 level based on a comparison of components of the emitted visible light reflected from the color change material.

In some embodiments according to the invention, the components include at least two color components of the emitted visible light reflected from the color change material. In some embodiments according to the invention, the at least two color components of the emitted visible light reflected from the color change material comprise red, green, and blue components.

In some embodiments according to the invention, the determining can be provided by determining the CO2 level based on a comparison of at least two of a red component, a green component, and a blue component of the emitted visible light reflected from the color change material.

In some embodiments according to the invention, an apparatus to monitor a respiratory stream can include a color change material that can be positioned proximate to the respiratory stream an electronic visible light emitter that can be configured to emit visible light onto the color change material.

In some embodiments according to the invention, the apparatus can include an electronic visible light sensor, that can be positioned to receive at least a portion of the emitted visible light reflected from the color change material. In some embodiments according to the invention, the electronic visible light emitter and the electronic visible light sensor are remote from the respiratory stream, and the apparatus can further include an optical transmission medium that extends from the color change material to the electronic visible light emitter and the electronic visible light sensor, that can be configured to conduct the emitted visible light onto the color change material and to conduct the emitted visible light reflected from the color change material.

In some embodiments according to the invention, the apparatus can further include a breathing circuit adapter having the color change material mounted on an interior side wall thereof, wherein a major surface of the color change material is parallel to a direction of the respiratory stream in the adapter.

In some embodiments according to the invention, a composition for use in monitoring a respiratory stream can include a color change material configured to change from a first color to a second color in response to an increase in CO2 within the respiratory stream, where the first color includes more of a first component than a second component or more than a third component and the second color includes less of the first component than the second component or less than the third component.

In some embodiments according to the invention, the first component can be blue and the second and third components can be red and green, respectively. In some embodiments according to the invention, the first color includes more of the first component than both the first and second components and the second color includes less of the first component than both the second and third components.

### BRIEF DESCRIPTION OF THE DRAWINGS

FIG. 1 is a schematic illustration of a color change material configured for placement within a breathing circuit for contact with CO₂ in some embodiments according to the invention.

FIG. 2 is a schematic representation illustrating a chemical reaction between a color change indicator included in the color change material and CO₂ in contact therewith as part of the breathing cycle in some embodiments according to the invention.

FIGS. 3-6 are schematic representations illustrating different configurations of color change materials in some embodiments according to the invention.

FIG. 7 is a schematic representation of a color change material included in a breathing circuit and exposed to electronically generated visible light and electronic sensing thereof in some embodiments according to the invention.

FIG. 8 is a schematic representation of a CO₂ detection system in some embodiments according to the invention.

FIG. 9 is a schematic representation of a CO₂ detection system in some embodiments according to the invention.

FIG. 10 is a schematic representation of a CO₂ detection system in some embodiments according to the invention.

FIG. 11 is a schematic representation of a CO₂ detection system in some embodiments according to the invention.

FIG. 12 is a schematic illustration of a display configured to provide information regarding CO₂ provided by the CO₂ system in some embodiments according to the invention.

FIG. 13 is a schematic illustration of a mask incorporating a display configured to provide CO₂ information provided by the CO₂ system in some embodiments according to the invention.

FIG. 14 is a schematic illustration of a CO₂ detection system utilized in an open breathing environment in some embodiments according to the present invention.

FIG. 15 is a greater detail schematic illustration of the CO₂ detection system shown in FIG. 14 in some embodiments according to the invention.

FIG. 16 is a schematic illustration of the CO₂ detection system including optical components in some embodiments according to the invention.

FIG. 17 is a schematic illustration of test setup for a CO₂ detection system in some embodiments according to the invention.

FIG. 18 is a graph illustrating CO₂ information generated by the CO₂ detection system operating in the test setup shown in FIG. 17.

FIG. 19 is a 1931 CIE chromaticity diagram.

### DETAILED DESCRIPTION OF EMBODIMENTS ACCORDING TO THE INVENTION

Embodiments of the present inventive subject matter now will be described more fully hereinafter with reference to the accompanying drawings, in which embodiments of the present inventive subject matter are shown. This present inventive subject matter may, however, be embodied in many different forms and should not be construed as limited to the embodiments set forth herein. Rather, these embodiments are provided so that this disclosure will be thorough and complete, and will fully convey the scope of the present inventive subject matter to those skilled in the art. Like numbers refer to like elements throughout.

It will be understood that in the embodiments discussed herein, the respiratory gasses can be those inhaled/exhaled by any living organism, such as a human, an animal, etc. Accordingly, the respiratory gas is referred to as being inhaled/exhaled by a subject, which can refer to any living organism.

In still further embodiments according to the invention, it will be understood that the use of the systems for the detection of CO₂ can be implemented in any environment where the measurement of CO₂ may be desirable. For example, in some embodiments according to the invention, systems, etc. for the detection of CO₂ as described herein may be implemented as part of mass transit systems (such as trains, airplanes, buses, etc.), places where large crowds congregate, such as stadiums etc., environments where the level of CO₂ in a subject undergoing physical exercise may be monitored, such as during running, training, or other physical exertion with a level of CO₂ expired by the subject may be relevant. In still other embodiments according to the invention, systems as described herein may be utilized to detect the level of CO₂ in closed breathing systems other than those normally associated with medical procedures, such as use with fire fighting breathing apparatus, mining environments, underwater breathing equipment (i.e., scuba), space applications, and military applications, etc.

In other embodiments according to the invention, the level of CO₂ associated with subject may be provided in environments such as emergency situations wherein CO₂ levels may be determined by first responders, where such first responders would utilize what is commonly referred to as an emergency CO₂ detector in connection with an endotracheal tube. In still other embodiments according to the invention, the level of CO₂ described herein may be determined in association with the administration of IV sedation, such as that used during dentistry or other medical procedures where full anesthesia is not required or used.

It will be understood that the levels of CO₂ using systems, devices, methods, etc. as described herein can be utilized in any system that employs a breathing circuit. Such environments may include a ventilator, a respirator, etc., which may be used in conjunction with the administration of anesthesia in an operating room, emergency room, etc. where a level of CO₂ may provide an accurate and relatively quick indication of heart/lung function and otherwise provide medical professionals with an indication of the patient's stability.

In some embodiments according to the invention, the CO₂ detection systems may be utilized in what is referred to as an open breathing environment, where the color change material included in the system is not housed within a tube of other enclosure through which the respiratory gas stream flows. Other types of environments and applications are also described herein.

Further, it will be understood that although many embodiments are described herein as using visible light from an electronic emitter, other types of light many be used to determine levels of CO₂ consistent with the inventive concepts described herein.

As appreciated by the present inventors, various existing CO₂ detection schemes may rely on a visual color change in a detector configured with colored paper responsive to CO₂ absorption. Such detectors can indicate the presence or absence of CO₂ in a respiratory stream, and are commonly used in emergency medical settings. However, these detectors generally do not provide sufficient accuracy to guide clinical decisions regarding effectiveness of emergency procedures such as ventilation and/or CPR. Moreover, such devices may have limitations with respect to working life once activated, since CO₂ absorption from the atmosphere or from the respiratory gas stream eventually exhausts the capacity of the absorber in the detector.

Embodiments according to the invention can provide for colorimetric detection of CO₂ in a stream of respiratory gases using electronically generated visible light and electronic detection of the colorimetric change. Accordingly, in some embodiments according to the present invention, a color change material can be placed in contact with the respiratory stream, such as when located on the interior wall of a portion of breathing circuit. A first surface of the color change material can be in contact with the interior wall while a second surface can be in contact with at least a portion of the respiratory stream.

Carbon dioxide gas within the respiratory stream may diffuse partially into the color change material (which includes a composition referred to as a color change indicator), where it may undergo absorption and/or reaction with components within the layer. Absorption and/or reaction within the layer may result in a color change of the indicator within the layer that is indicative of the amount of CO₂ absorbed by the layer and thereby may provide an indication of CO₂ in the respiratory stream. The color change material may be configured to permit rapid absorption and desorption of CO₂ in order to facilitate sensing of a time-varying level of CO₂ in the respiratory stream and may be reversible in that variation of the CO₂ is indicated as the gas is exhaled/inhaled.

Respiratory gas flow may be confined within, for example, a tube that makes up part of the breathing circuit. The color change material can be located in any portion of the interior of the tube and oriented to allow the respiratory stream to flow across the major surface of the material. An electronic emitter can provide a visible light source with suitable color output may be positioned outside the tube, such that a portion of emitted light is projected through the wall of the tube to illuminate the color change material. An electronic sensor can detect the color change exhibited by the color change layer, which can then be used to indicate the level of CO₂ in the respiratory stream.

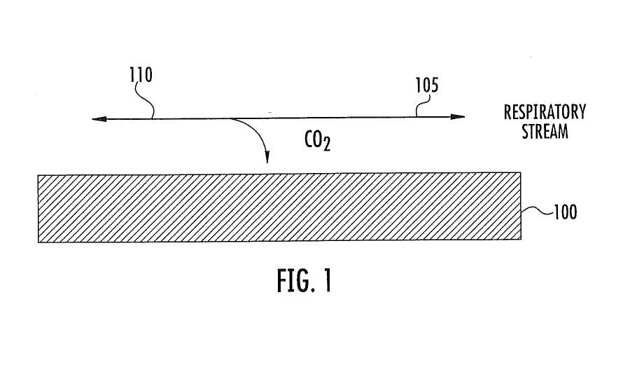

FIG. 1 is a schematic illustration of a color change material 100 that is configured for inclusion within a breathing circuit in some embodiments according to the invention. According to FIG. 1, the color change material 100 is configured for contact with a subject's respiratory stream. The color change material 100 is positioned within the stream so that when the subject exhales, exhaled gas contacts the major surface of the color change material 100 in the first direction 105. When the subject inhales, inhalation gas is drawn across the major surface of the color change material 100 in the direction 110 which is generally opposite to the direction 105.

It will be understood that the generation of the exhalation gas in the direction 105 and the inhalation gas in the direction 110 is generally referred to herein as a cycle of breathing (i.e., cycle) and further that the exhalation 105 and the inhalation 110 are referred to together as a respiratory gas. It will be further understood that portions of the respiratory gas can flow in other directions which are not parallel to the major surface of the color change material 100. It will be further understood that the color change material 100 is positioned within the breathing circuit so that the respiratory gas is drawn across the major surface of the color change material 100 during the breathing cycle in a repeatable and consistent fashion. Accordingly, the orientation of the color change material 100 within the breathing circuit can reduce obstruction to the respiratory gas. For example, such configurations of the color change material 100 within the breathing circuit can be provided when, for example, the color change material 100 is placed “in-line” in an endotracheal tube or near an exit port of a face mask (such as a mask used for the administration of anesthesia), or in-line with a spirometer, etc.

The color change material 100 shown in FIG. 1 can include a color change indicator configured for detection and measurement of the level of carbon dioxide in the respiratory stream using a reversible color change in response to the presence of carbon dioxide. It will be understood that the color change indicator can be a composition that is impregnated or otherwise included in the color change material 100.

In some embodiments according to the invention, the color change indicator can include an alkaline material reactive to gaseous carbon dioxide and may thereby change the pH of a portion of the color-change layer in contact with a respiratory stream containing carbon dioxide. Exemplary alkaline materials may include sodium carbonate, potassium carbonate, calcium carbonate, magnesium carbonate, sodium hydroxide, potassium hydroxide, primary, secondary, or tertiary amines, or combinations thereof. In some embodiments according to the invention, the color change indicator can include a dye or pigment that may undergo reversible color change in response to change in pH. Exemplary indicators may include metacresol purple, thymol blue, and phenol red, and combinations thereof. In some embodiments according to the invention, the color change indicator may include two or more dyes or pigments.

In some embodiments according to the invention, the color change indicator can include one or more buffers to modify the pH of the color-change layer. Buffers may also be selected to provide faster response time, better reversibility, and longer life. Exemplary buffers include aqueous solutions of sodium bisulfate, sodium carbonate, and mixtures thereof In some embodiments according to the invention, the color change indicator can be configured to undergo a change in color and/or color saturation in the presence of a metabolically relevant carbon dioxide concentration. In some embodiments according to the invention, the color change indicator comprises an alkaline material, a dye or pigment, and one or more buffers.

In some embodiments according to the invention, the color change indicator can include a water-attractive component to facilitate hydration of the color-change layer in the presence of vapor-phase moisture in the respiratory stream. Exemplary water-attractive components may include glycerol, propylene glycol and mixtures thereof. In some embodiments according to the invention, the color change indicator comprises an alkaline material, a dye or pigment, one or more buffers, and a water-attractive component. In some embodiments according to the invention, the color change indicator can include surface modifying additives including ionic and nonionic surfactants. Exemplary surfactants include, but are not limited to, amines, such as mono-, di-, and trimethanolamine, and quaternary ammonium compounds, such as benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide. In some embodiments according to the invention, the color change indicator can include an antimicrobial additive to inhibit growth of bacteria, molds, funguses or other microbes. As appreciated by the present inventors, in some embodiments, the presence of an amine and/or quaternary ammonium compound in the color change indicator may increase the magnitude of the color change and/or color saturation when in the presence of a metabolically relevant carbon dioxide concentration.

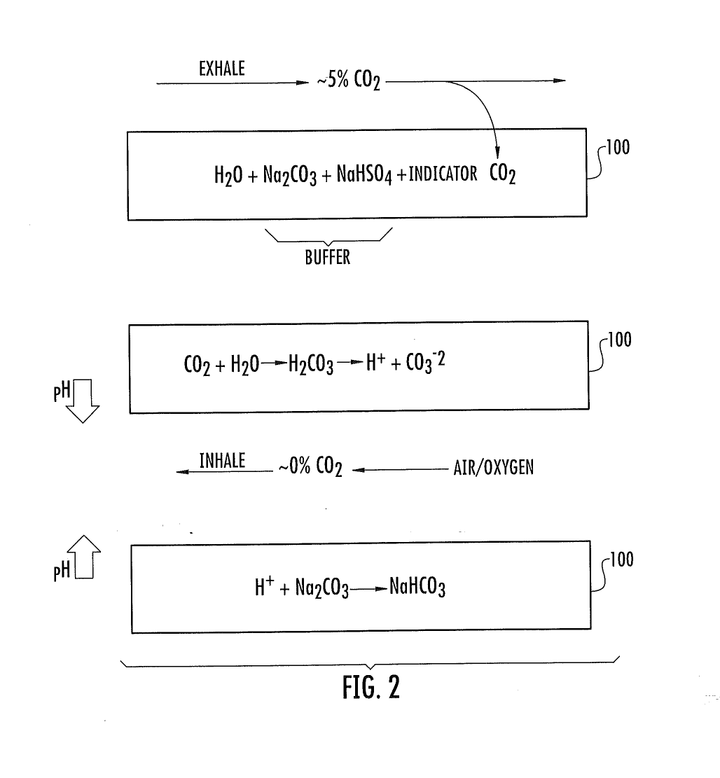

FIG. 2 is a schematic representation of operation of the color change indicator in the color change material 100 responsive to respiratory gas during a breathing cycle in some embodiments according to the invention. According to FIG. 2, respiratory gas including about 5% CO₂ contacts the color change material 100. It will be understood that in some embodiments according to the invention, the color change material 100 includes a buffer as well as the color change indicator described herein. According to FIG. 2, the buffer can include Na₂CO₃ and NaHSO₄ together which operate to stabilize the pH of the color change material 100. Water (H₂O) can also be introduced into the color change material 100 via moisture carrier in the respiratory gas during the exhalation portion of the cycle. It will be understood that the pH exhibited by the color change indicator in an initial condition (i.e., prior to the exhalation cycle and the absorption of CO₂) can be at a pH from about 7 to about 14, or any range therein, such as, from about 7 to about 12, or from about 8 to about 10. In some embodiments according to the invention, the color change indicator can be at a pH of about 9.

During the exhale cycle, a portion of the CO₂ is absorbed into the color change material 100, whereupon the carbon dioxide and water react to create H₂CO₃ whereupon a hydrogen ion (H+) becomes disassociated therewith and also generates the byproducts shown. Because the CO₂ is in a gaseous form, the carbon dioxide can diffuse into the color change material 100 faster than the buffer may be able to stabilize the pH so that the hydrogen ions lower the pH of the color change material 100, such that the color exhibited by the color change indicator shifts.

As shown in FIG. 2, during the inhale portion of the breathing cycle, time elapses where no CO₂ is introduced into the color change material 100 so the time is provided for the hydrogen ions to combine with the base portion of the buffer to again raise the pH of the color change material 100 to the static condition (e.g., about a pH of 9). It will be understood that the above described breathing cycle is then repeated as the subject continues to breathe. It will be further understood that the amount of the buffer introduced into the color change material 100 can be configured to allow the color change material 100 to exhibit the color change for the desired period of time whereupon the buffer may be replenished for further operation.

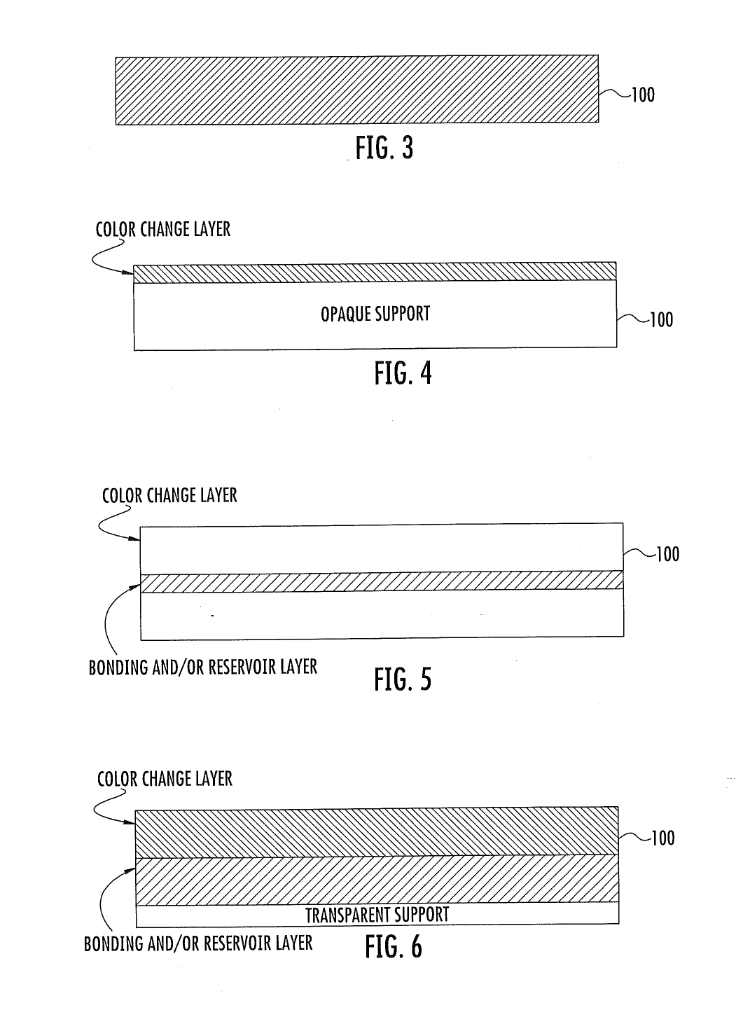

FIGS. 3-6 are schematic illustrations of different configurations of a color change material 100 allowing for different applications in some embodiments according to the invention. In particular, in some configurations the color change material can include a thin material, such as paper, having the color change indicator infused therein. In other embodiments, a separate substrate may be provided to which the color change material is attached. In still other embodiments, the color change material can be support by what is referred to a mineral support, which can allow the color change indicator to be applied in the form of a composition onto to a surface of the breathing circuit in some embodiments according to the invention.

In some embodiments according to the invention, the color change material 100 can be provided in the form of a unitary format, such as a liquid including color change indicator (which may be, for example sprayed or painted onto a surface) or the color change indicator impregnated into a substrate such as a thin paper. Accordingly, in these embodiments according to the invention, the color change material 100 can be painted or coated onto an interior surface of the breathing circuit. Accordingly, the color change material 100 can include unitary layer with high specific surface area. The unitary layer may be impregnated with chemical species that bring about a reversible color change in response to carbon dioxide in the respiratory stream. The unitary layer may be porous or microporous. Exemplary unitary layers include cellulosic paper, microporous olefinic synthetic paper, and various coatings based on particulates such as clay and/or silica and/or ground limestone and/or purlite and/or talc or other mineral-based materials. Other coatings may contain finely divided cellulose and/or other finely divided organic materials or combinations thereof.

In some embodiments according to the invention, the color change material 100 is a multilayer construction comprising a substrate, a bonding layer, and a color-change layer (including the color change indicator). See, for example, FIGS. 4-6. The substrate may be selected from a variety of thin, rigid or flexible materials such as paper, glass, or plastic films or sheets, or molded plastic articles. Substrate materials may be optically transparent, reflective, or opaque, or some combination thereof The substrate material may be selected in order to provide mechanical support for a color-change layer, and also may be selected to have desirable optical properties such as transmission, reflectance, or opacity, to facilitate photometric measurement of the color-change layer. A bonding layer may be applied to the substrate to adhesively attach the color-change layer. The bonding layer may be selected for good mechanical bonding between the color-change layer and the substrate. The bonding layer may further be selected to provide a source of chemical agents that facilitate the color-change chemistry by migration of said agents from the bonding layer into the color-change layer. A color-change layer may be included that has a high specific surface area to facilitate interaction with a respiratory stream. The color change layer may be porous or microporous. The color-change layer may be impregnated with chemical species that bring about a reversible color change in response to carbon dioxide or other exhaled gases in the respiratory stream.

In some embodiments according to the invention, the color change material 100 can be provided as shown for example in FIG. 5, wherein the color change material 100 is a multilayer construction comprising a substrate, a bonding layer, and a color-change layer (including the color change indicator). In this embodiment, the substrate may be a portion of the airway circuit containing at least a portion of a respiratory stream. A bonding layer may be applied to the substrate to adhesively attach the color-change layer. The bonding layer may be selected for good mechanical bonding between the color-change layer and the substrate. The bonding layer may further be selected to provide a source of chemical agents that facilitate the color-change chemistry by migration of said agents from the bonding layer into the color-change layer. A color-change layer may be included that has a high specific surface area to facilitate interaction with a respiratory stream. The color change layer may be porous or microporous. The color-change layer may be impregnated with chemical species that bring about a reversible color change in response to carbon dioxide in the respiratory stream.

In some embodiments according to the invention, as shown for example in FIG. 6, the color change material 100 is a substantially transparent article, such as a planar waveguide, with a color-change layer adhesively attached to at least one edge of the waveguide, and wherein the portion of the waveguide having a color-layer attached thereto is projected into a portion of a respiratory stream.

As described herein, the color change material 100 can include a color change indicator, which may be incorporated into the color change material 100 structures shown in FIGS. 4-6, for example, as a color change layer. The color change indicator can provide for the colorimetric response in the presence of CO2. The following examples describe exemplary color change indicators that were fabricated:

### EXAMPLE 1

A color change indicator according to the present invention was fabricated using 0.4 grams of anhydrous sodium bisulfate dissolved in 9.6 grams of water. 5.0 grams of glycerin was added and mixed to dissolve. 1.0 gram of a 0.1% w/w aqueous solution of metacresol purple dye was added and stirred to mix, resulting in a red colored solution. A 10% w/w aqueous solution of anhydrous sodium carbonate was added drop-wise until the color of the solution permanently changed to purple, occurring at a pH of approximately 9.0.

### EXAMPLE 2

Another color change indicator according to the present invention was fabricated using 0.5 grams of anhydrous sodium bisulfate were dissolved in 9.5 grams of water. 5.0 grams of glycerin was added and mixed to dissolve. 1.0 gram of a 0.1% w/w aqueous solution of metacresol purple dye was added and stirred to mix, resulting in a red colored solution. A 10% w/w aqueous solution of anhydrous sodium carbonate was added drop-wise until the color of the solution permanently changed to purple, occurring at a pH of approximately 9.0.

### EXAMPLE 3

A mineral support embodiment as an alternative to the impregnation of paper with the color change indicator was fabricated using 4.0 grams of kaolin clay combined with 2.0 grams of diatomaceous earth (Celite 535), 3.0 grams water, and 1.0 gram of Neocryl A-614 acrylic latex resin (DSM Neoresins) to form a stiff paste. A layer approximately 3 mils in thickness was doctor-bladed onto a heavy poster-paper support and baked in an oven for 5 minutes at 150° C. The resulting layer was nearly white in color, adherent, and had a matte finish.

### EXAMPLE 4

A mineral support was fabricated using 1.0 grams of kaolin clay combined with 5.0 grams of calcium carbonate, 3.0 grams of water, and 1.0 gram of Neocryl A-614 acrylic latex resin (DSM Neoresins) to form a stiff paste. A layer approximately 3 mils in thickness was doctor-bladed onto a heavy poster-paper support and baked in an oven for 5 minutes at 150° C. The resulting layer was nearly white in color, opaque, adherent, with a matte finish.

### EXAMPLE 5

An embodiment of the color change material 100 shown in FIG. 6 was fabricated using a sheet of polycarbonate plastic approximately 30 mils in thickness laminated to a sheet of white paper having a basis weight of approximately 270 g/square meter using an adhesive layer consisting of 3.0 grams of a 10% (w/w) solution of monoethanolamine in methanol and 5.0 grams of Neocryl A-614 acrylic latex resin (DSM Neoresins). The laminated construction was baked in an oven at 100° C. for 5 minutes. The resulting construction had an adherent white paper layer firmly attached to a transparent polycarbonate support layer.

### EXAMPLE 6

A change color material 100 shown in the embodiment illustrated in FIG. 3 was fabricated using strips of conventional ink-jet printer paper approximately 1 inch wide and 2 inches long were soaked in the color change of examples 1 or 2 indicator for 5-10 seconds, drained on absorbent toweling, and baked at about 100° C. for 60 sec. The resulting paper strips had an intense purple color on both sides, were dry to the touch, and spontaneously and reversibly changed in color shade when exposed to physiologically relevant levels of carbon dioxide, e.g. 1-10% (v/v) in air at approximately one atmosphere pressure. Color shade variation in response to carbon dioxide was discernible from either side of the strip.

### EXAMPLE 7

A color change material 100 according to the embodiment illustrated in FIG. 3 was fabricated using strips of mineral support of examples 3 and 4 approximately 1 inch wide and 2 inches long were soaked for 5-10 seconds in Color Change Indicator 2, and baked in an oven at 100° C. for 60 sec. The resulting strips were opaque, had an intense purple color on the mineral-coated side, were dry to the touch, and spontaneously and reversibly changed in color shade when exposed to physiologically relevant levels of carbon dioxide, e.g. 1-10% v/v in air at approximately one atmosphere pressure.

### EXAMPLE 8

A color change material 100 illustrated in FIG. 6 are fabricated using strips of the plastic support of example 5 1 approximately 1 inch wide and 2 inches long were soaked for 5-10 seconds in color change Indicator of examples 1 or 2, and baked in an oven at 100° C. for 60 sec. The resulting strips had an intense purple color, were partially transparent, were dry to the touch, and spontaneously and reversibly changed in color shade when exposed to physiologically relevant levels of carbon dioxide, e.g. 1-10% v/v in air at approximately one atmosphere pressure. The color shade variation was discernible from either side of the plastic support.

### EXAMPLE 9

A color change indicator according to embodiments of the present invention was prepared by dissolving 0.44 gram of anhydrous sodium bisulfate in 9.0 grams of water, adding 5.0 grams of glycerol, stirring to mix, then adding 1.0 gram of an aqueous 0.1% (w/w) solution of metacresol purple. The solution was titrated to a permanent grape-purple color with approximately 1.67 grams of an aqueous 20%(w/w) solution of sodium carbonate monohydrate. Twenty parts by volume of the resulting solution were combined with 2 parts by volume of a solution of benzalkonium chloride (Andwin Scientific part number 190009) and 3 parts by volume of a 10 % (w/w) solution of monoethanolamine in methyl alcohol. The resulting solution was brushed onto strips of white paper having a basis weight of approximately 320 grams per square meter, then baked in an oven for 60 seconds at approximately 100 degrees C. The resulting strips had a uniform sky-blue color, were dry to the touch, and spontaneously and reversibly changed in color shade when exposed to physiologically relevant levels of carbon dioxide, e.g. 1-10% v/v in air at approximately one atmosphere pressure. The color shade variation was discernible from either side of the strip.

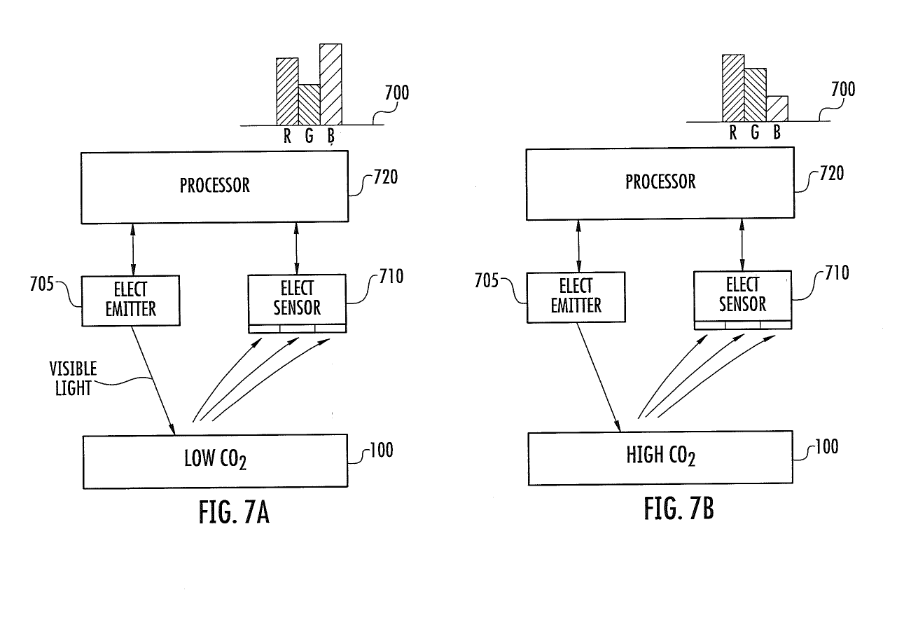

FIGS. 7A and 7B are schematic illustrations of a CO₂ detection system in some embodiments according to the invention. In particular, FIG. 7A illustrates operation of the CO₂ detection system 700 where the color change material 100 is exposed to a relatively low concentration of CO₂, such as when a subject inhales as part of the breathing cycle. At this time, the electronic light emitter 705 emits visible light to illuminate the color change material 100 which is detected by an electronic light sensor 710, both of which can operate under the control of a processor 720. In some embodiments according to the invention, visible light includes light that falls within a range of wavelength of about 400 nm to about 700 nm, so that at least some of this range may not be perceptible to a human observer without the assistance of embodiments according to the invention.

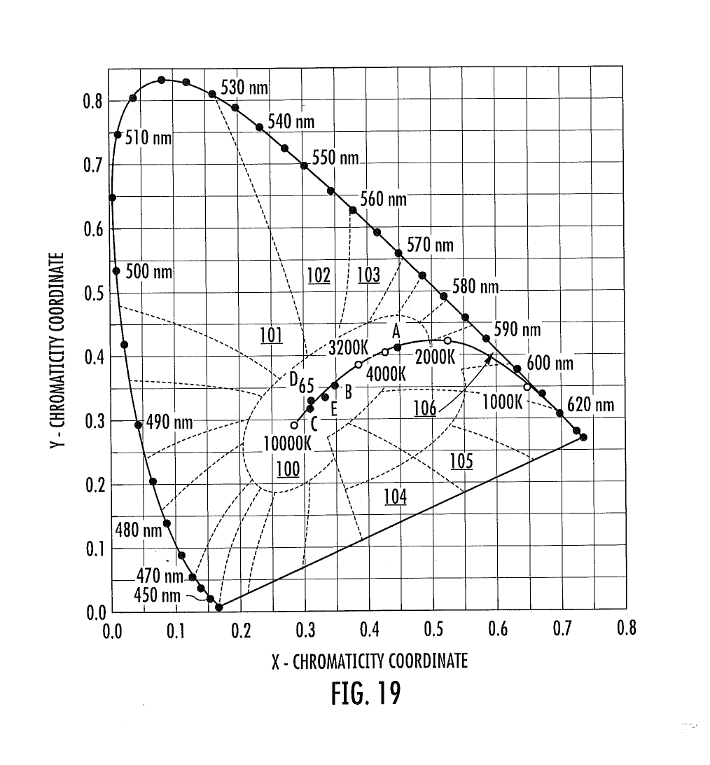

As describe herein, during the inhale portion of the breathing cycle, the relatively low concentration of CO₂ in the respiratory stream causes little or no change in the pH of the color change indicator 100 and pH remains generally constant at approximately pH 9. No color shift occurs in the indicator 100 and the reflected light detected by the electronic sensor 700 has a particular value similar in magnitude to the initial color of the color indicator. For example, in some embodiments according to the invention, the value of the reflected light detected by the electronic sensor 700 can be separated into its color components, such as red, green and blue components of the visible light, each of which may be characterized by a particular value, such as an intensity, color value, color temperature etc. In other embodiments according to the invention, the components of the visible light may represent a single color temperature value, which can be represented using, for example, the 1931 CIE chart shown in FIG. 19, The value of the light reflected from the color change indicator 100 and detected by the electronic sensor 700 can indicate the level of CO₂ that contacts the color change indicator 100, which can be determined by the processor 720.

FIG. 7B illustrates the same CO₂ detector system 700 operating during the exhale portion of the breathing cycle. According to FIG. 7B, the electronic emitter 705 emits visible light to illuminate the color change indicator 100 that is exposed to a relatively high concentration of CO₂ during the exhale portion of the breathing cycle. Accordingly, the increased concentration of CO₂ in contact with the color change indicator 100 can cause the pH of the color change indicator 100 to decrease (therefore becoming more acidic) which may, in turn, be reflected by a change in color of the color change indicator 100. This change in color can be detected by the electronic sensor 700 which can be represented using the same approach described above in reference to FIG. 7A. Therefore, as the breathing cycle proceeds, the change in the pH of the color change indicator 100 (due to the varying levels of CO₂ exposed thereto) can be determined by the electronic sensor 700 by analyzing the values of the reflected light detected by the electronic sensor 700.

In some embodiments according to the invention, “white” light can be used as the visible light, which includes components of red, green, and blue. Further, a ratio of the red component to the blue component (in the reflected light) may yield a first value of red-to-blue ratio when the color change indicator 100 is exposed to a relatively low concentration of CO₂. As further shown in FIG. 7A, the ratio of the green component to the blue component may also yield an initial first value of green-to-blue ratio in the same situation. It will be further understood that other types of visible light and components thereof may also be utilized.

In contrast, as shown in FIG. 7B, when the color change indicator 100 is exposed to the relatively high concentration of CO₂, the ratio of the red component to the blue component may yield a second value that is greater than the first value. As further shown in FIG. 7B, a ratio of the green component to the blue component is also greater than the first value. As appreciated by the present inventors, in some embodiments according to the invention, the green to blue ratio may be less susceptible to noise and to other external factors which can provide a more stable indication of color values detected in the environments illustrated by FIGS. 7A and 7B.

According to FIGS. 7A and 7B, the ratio of one component to another can increase in presence of increased levels of CO2. For example, in FIG. 7A, a relatively low level of CO2 can be evidenced by red, green, and blue color components 80, 50, and 70, respectively. When, however, the level of CO2 increases, as illustrated in FIG. 7B, the color component values can change to, for example, 83, 55, and 71, respectively (where the component values are expressed as values/100). Therefore, a change in the ratio of selected components to one another can indicate the change in CO2.

In some embodiments according to the present invention, a comparison between multiple component values can provide the indication of CO2 levels. In some embodiments according to the invention, a change in a single component value can indicate a change in the CO2 level.

In some embodiments according to the invention, the color change material can be analyzed by selecting a first color or group of colors that become more saturated in the presence of CO2, a second color or group of colors that become less saturated in the presence of CO2, and a third color or group of colors whose saturation is insensitive to the presence of CO2. A scaling factor can be determined for each of the first, second, and third colors and a computational method can be applied to combine the first, second, and third colors and/or their respective scaling factors in order to compute a value representative of the CO2 concentration in the colorimetric sensor, such that the CO2 concentration thereby calculated is relatively insensitive to interference effects from moisture, condensation, or long-term color drift caused by depletion of buffer in the colorimetric sensor material.

In some embodiments according to the invention, the first color or group of colors may be selected to coincide with one or more absorption maxima in the absorption spectra of the at least partially deprotonated indicator dye. In some embodiments according to the invention, the second color or group of colors may be selected to coincide with one or more absorption minima in the absorption spectra of the at least partially protonated indicator dye.

In some embodiments according to the invention, the third color or group of colors may be selected to coincide with one or more isobestic points in the absorption spectrum of the color indicating dye. In some embodiments according to the invention, the first and second colors or groups of colors may be selected on the basis of computing a maximum signal level in the detector response, regardless of where the colors may fall in the absorption spectrum. In some embodiments according to the invention, an instant ratio of color saturation of colors from the first and second color groups is compared with a time-weighted and/or running average of the color saturation of the first and second color groups. The electronic emitter 720 can be a light emitting device, such as a light emitting diode, along with other support electronics used to operate the LED using the processor 720, such as a driver circuit to provide biasing and current to the LED(s).

A representative example of a white LED lamp includes a package of a blue light emitting diode chip, made of gallium nitride (GaN), coated with a phosphor such as YAG. In such an LED lamp, the blue light emitting diode chip produces a blue emission and the phosphor produces yellow fluorescence on receiving that emission, which is sometimes referred to as blue-shifted-yellow (BSY). For instance, white light emitting diodes can be fabricated by forming a ceramic phosphor layer on the output surface of a blue light-emitting semiconductor light emitting diode. Part of the blue ray emitted from the light emitting diode chip passes through the phosphor, while part of the blue ray emitted from the light emitting diode chip is absorbed by the phosphor, which becomes excited and emits a yellow ray. The part of the blue light emitted by the light emitting diode which is transmitted through the phosphor is mixed with the yellow light emitted by the phosphor.

More specifically, a “BSY LED” refers to a blue LED and an associated recipient luminophoric medium that together emit light having a color point that falls within a trapezoidal “BSY region” on the 1931 CIE Chromaticity Diagram (FIG. 19) defined by the following x, y chromaticity coordinates: (0.32, 0.40), (0.36, 0.48), (0.43, 0.45), (0.42, 0.42), (0.36, 0.38), (0.32, 0.40), which is generally within the yellow color range, see for example, FIG. 5. A “BSG LED” refers to a blue LED and an associated recipient luminophoric medium that together emit light having a color point that falls within a trapezoidal “BSG region” on the 1931 CIE Chromaticity Diagram defined by the following x, y chromaticity coordinates: (0.35, 0.48), (0.26, 0.50), (0.13, 0.26), (0.15, 0.20), (0.26, 0.28), (0.35, 0.48), which is generally within the green color range. A “BSR LED” refers to a blue LED that includes a recipient luminophoric medium that emits light having a dominant wavelength between 600 and 720 nm in response to the light emitted by the blue LED. A BSR LED will typically have two distinct spectral peaks on a plot of light output versus wavelength, namely a first peak at the peak wavelength of the blue LED in the blue color range and a second peak at the peak wavelength of the luminescent materials in the recipient luminophoric medium when excited by the light from the blue LED, which is within the red color range. Typically, the red LEDs and/or BSR LEDs will have a dominant wavelength between 600 and 660 nm, and in most cases between 600 and 640 nm.

As shown in FIG. 19, colors on the 1931 CIE Chromaticity Diagram are defined by x and y coordinates (i.e., chromaticity coordinates, or color points) that fall within a generally U-shaped area. Colors on or near the outside of the area are saturated colors composed of light having a single wavelength, or a very small wavelength distribution. Colors on the interior of the area are unsaturated colors that are composed of a mixture of different wavelengths. White light, which can be a mixture of many different wavelengths, is generally found near the middle of the diagram, in the region labeled 100 in FIG. 5. There are many different hues of light that may be considered “white,” as evidenced by the size of the region 100. For example, some “white” light, such as light generated by sodium vapor lighting devices, may appear yellowish in color, while other “white” light, such as light generated by some fluorescent lighting devices, may appear more bluish in color.

Light that generally appears green is plotted in the regions 101, 102 and 103 that are above the white region 100, while light below the white region 100 generally appears pink, purple or magenta. For example, light plotted in regions 104 and 105 of FIG. 5 generally appears magenta (i.e., red-purple or purplish red).

Also illustrated in FIG. 5 is the planckian locus 106, which corresponds to the location of color points of light emitted by a black-body radiator that is heated to various temperatures. In particular, FIG. 5 includes temperature listings along the black-body locus. These temperature listings show the color path of light emitted by a black-body radiator that is heated to such temperatures. As a heated object becomes incandescent, it first glows reddish, then yellowish, then white, and finally bluish, as the wavelength associated with the peak radiation of the black-body radiator becomes progressively shorter with increased temperature. Illuminants which produce light which is on or near the black-body locus can thus be described in terms of their correlated color temperature (CCT).

The chromaticity of a particular light source may be referred to as the “color point” of the source. For a white light source, the chromaticity may be referred to as the “white point” of the source. As noted above, the white point of a white light source may fall along the planckian locus. Accordingly, a white point may be identified by a correlated color temperature (CCT) of the light source. White light typically has a CCT of between about 2000 K and 8000 K. White light with a CCT of 4000 may appear yellowish in color, while light with a CCT of 8000 K may appear more bluish in color. Color coordinates that lie on or near the black-body locus at a color temperature between about 2500 K and 6000 K may yield pleasing white light to a human observer.

“White” light also includes light that is near, but not directly on the planckian locus. A Macadam ellipse can be used on a 1931 CIE Chromaticity Diagram to identify color points that are so closely related that they appear the same, or substantially similar, to a human observer. A Macadam ellipse is a closed region around a center point in a two-dimensional chromaticity space, such as the 1931 CIE Chromaticity Diagram, that encompasses all points that are visually indistinguishable from the center point. A seven-step Macadam ellipse captures points that are indistinguishable to an ordinary observer within seven standard deviations, a ten step Macadam ellipse captures points that are indistinguishable to an ordinary observer within ten standard deviations, and so on. Accordingly, light having a color point that is within about a ten step Macadam ellipse of a point on the planckian locus may be considered to have the same color as the point on the planckian locus.

The use of these types (and other) LEDs can promote truer color reproduction, which is typically measured using the Color Rendering Index (CRI). CRI is a relative measurement of how the color rendition of an illumination system compares to that of a blackbody radiator, i.e., it is a relative measure of the shift in surface color of an object when lit by a particular lamp. The CRI equals 100 if the color coordinates of a set of test colors being illuminated by the illumination system are the same as the coordinates of the same test colors being irradiated by the blackbody radiator. Daylight has the highest CRI (of 100), with incandescent bulbs being relatively close (about 95), and fluorescent lighting being less accurate (70-85). Certain types of specialized lighting have relatively low CRI's (e.g., mercury vapor or sodium, both as low as about 40 or even lower). Sodium lights are used, e.g., to light highways. Driver response time, however, significantly decreases with lower CRI values (for any given brightness, legibility decreases with lower CRI). Accordingly, the processor 720 can utilize, for example, CRI, color temperature, color values, CCT, etc. to determine values associated with the reflected light received by the electronic sensor 710, which can in turn be used to determine a CO₂ level. It will be understood that the CO₂ level can be determined by any approach, such as an equation or lookup table.

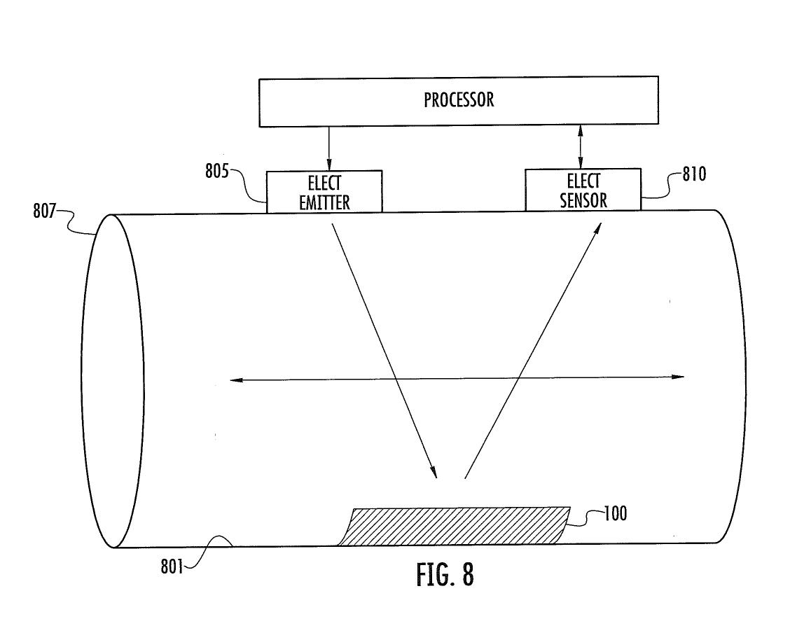

FIG. 8 is a schematic representation of a CO₂ detection system in some embodiments according to the invention. As shown in FIG. 8, the color change material 100 is located on an interior sidewall 801 of an adapter 807 configured to be removably coupled to a breathing circuit. For example, the adapter 807 is configured to be removably coupled to standard form-factor tubing typically used in systems such as ventilators, respirators, and other equipment used for medical procedures such as in operating rooms, emergency rooms, etc. The adapter 807 is further configured to allow the respiratory stream to flow longitudinally so that at least a portion of the respiratory gas conducted through the adapter 807 comes into contact with the surface of the color change material 100. It will be understood that due to the orientation and location of the color change material 100, the flow of respiratory gas is substantially unobstructed. Although the color change material 100 is shown attached to the sidewall 801, it will be understood that the color change material 100 can be located at any position within the interior of the adapter 807 while being longitudinally oriented as shown relative to the respiratory gas flow so as not to substantially impede the flow thereof.

An electronic emitter 805 is located outside the adapter 807 and is configured to emit visible light into the adapter 807 to illuminate the color change material 100 located on the adapter 807. An electronic sensor 810 is also located outside the adapter 807 and is configured to receive a portion of the light reflected by the color change material 100. As described herein, the change in the amount of CO₂ in the respiratory gases can cause a change in the pH of the color change material 100 thereby causing a shift in the color which can be detected using the electronic sensor 810 to determine the level of various light components of the visible light reflected by the color change material 100.

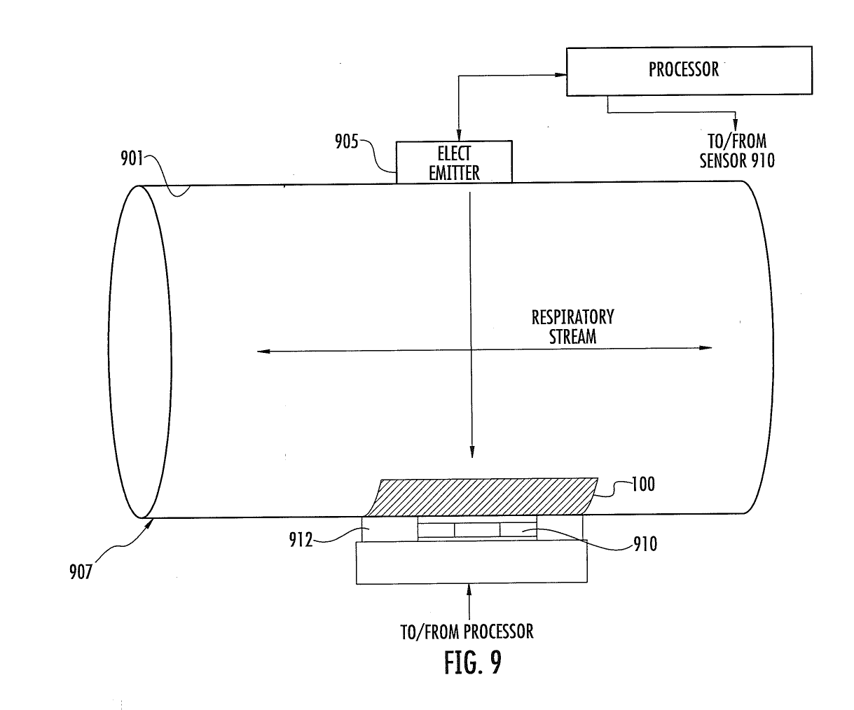

FIG. 9 is a schematic illustration of a CO₂ detection system in some embodiments according to the invention. According to FIG. 9, the color change material 100 is located on an interior surface 901 of an adapter 907. An electronic emitter 905 is located outside the adapter 907 opposite the color change material 100. The adapter 907 is configured to allow the respiratory gases to be conducted in a longitudinal direction while coming into contact with the surface of the color change material 100.

An electronic sensor 910 is located outside the adapter 907 behind the color change material 100 relative to the position of the electronic emitter 905. The electronic sensor 910 can be spaced apart from the outside surface of the adapter 907 by a spacer 912, which creates a space between a mounting for the sensor 910 and the surface. The space can be utilized to also accommodate filters (such as red, green, and blue filters) on the sensor 910, which can be used to promote the detection of those light components.

Accordingly, when the electronic emitter 905 emits visible light, the visible light impacts the color change material 100 but rather than reflecting from the surface to the sensor as described above in reference to, for example, FIG. 8, the visible light is detected by the electronic sensor 910 located on the opposing side of the color change material 100 on the outside of the adapter 907. It will be understood that the electronic sensor 910 can be used to determine the relative levels of CO₂ in the respiratory stream as described herein.

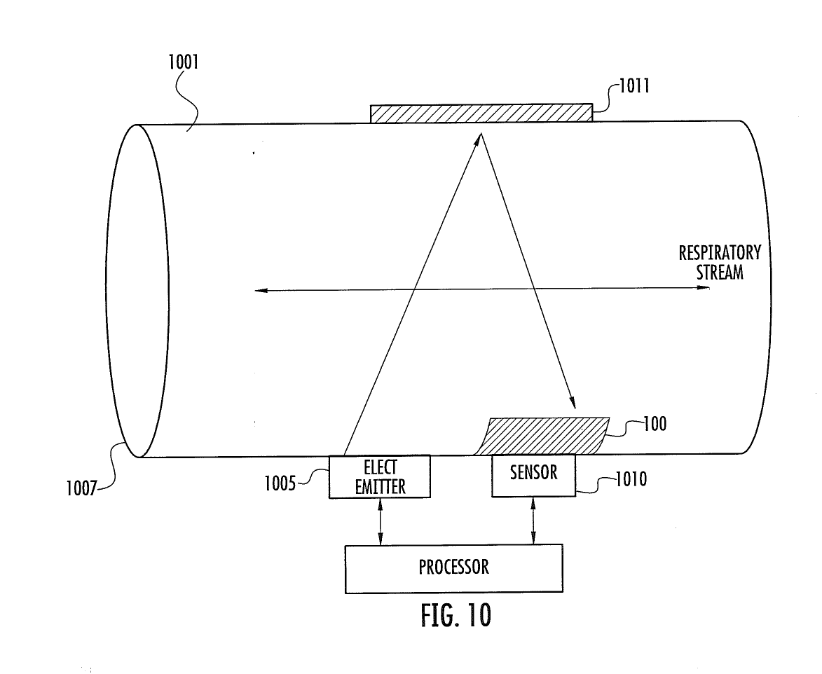

FIG. 10 is a schematic illustration of a CO₂ detection system in some embodiments according to the invention. According to FIG. 10, the color change material 100 is located on an interior surface 1001 of an adapter 1007 and is configured to allow the respiratory stream of gases to come into contact therewith without substantially restricting the flow thereof. As further shown in FIG. 10, a reflector 1011 is located outside the adapter 1007 on an opposing side thereof relative to the color change material 1100. An electronic emitter 1005 located outside the adapter 1007 and emits visible light to impact the reflector 1011 which is reflected onto the color change material 1100 as shown. The visible light reflected onto the color change material 100 is detected using an electronic sensor 1010 located outside the adapter 1007 on an opposing side thereof relative to the reflector 1011. It will be understood that the relative levels of CO₂ in the respiratory gas stream can be determined as described herein.

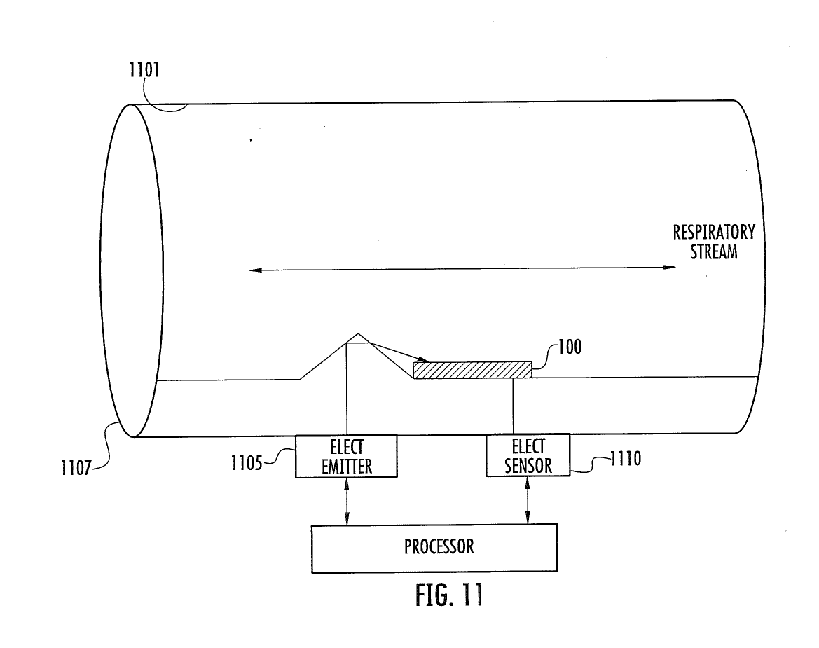

FIG. 11 is a schematic illustration of a CO₂ detection system in some embodiments according to the invention. According to FIG. 11, a color change material 100 is located on an interior surface 1101 of an adapter 1107. The color change material 100 is configured within the adapter 1107 to allow the respiratory gas stream conducted therein to come into contact therewith while not substantially obstructing the flow of respiratory gases. As further shown in FIG. 11, the sidewall of the adapter 1107 includes an optical path configured to refract visible light emitted by an electronic emitter 1105 onto the surface of the color change material 100. The visible light reflected onto the color change material 100 can be detected by an electronic sensor 1110. It will be understood that the relative levels of CO₂ in the respiratory gas stream can be determined based on the approaches described herein.

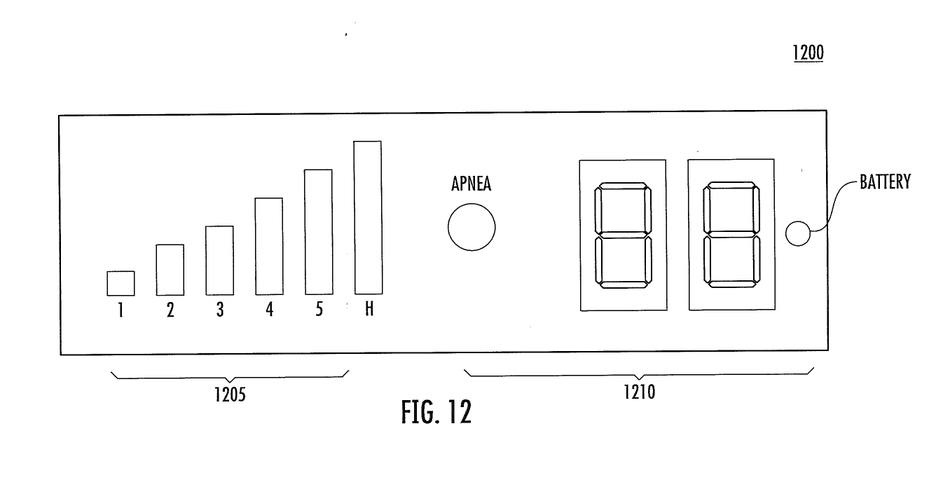

FIG. 12 is a schematic representation of an exemplary display included in a CO₂ detection system in some embodiments according to the invention. According to FIG. 12, a CO₂ level portion of the display 1205 indicates the level of CO₂ in the respiratory stream based on the electronic sensors processing of the color components included in the reflected visible light. An auxiliary portion of display 1210 can include other information regarding the status of the subject. For example, auxiliary information 1210 may include a read out RR which indicates respiration rate, an indicator light signaling an apnea condition, and a battery level indicator.

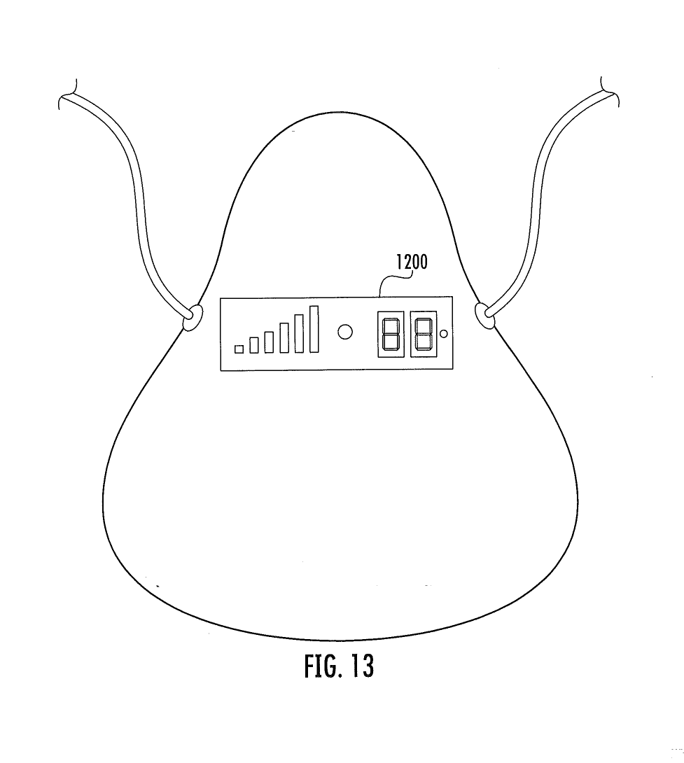

FIG. 13 is a schematic representation of a mask configured for placement over a subject's mouth and nose and including the display 1200 shown in FIG. 12, Although the display 1200 is shown located at a bridge portion of the mask, it will be understood that the display 1200 can be located in any orientation or location of the mask which facilitates its use in a particular environment. In particular, for example, in some embodiments according to the invention, the display 1200 may be located on a side portion of the mask.

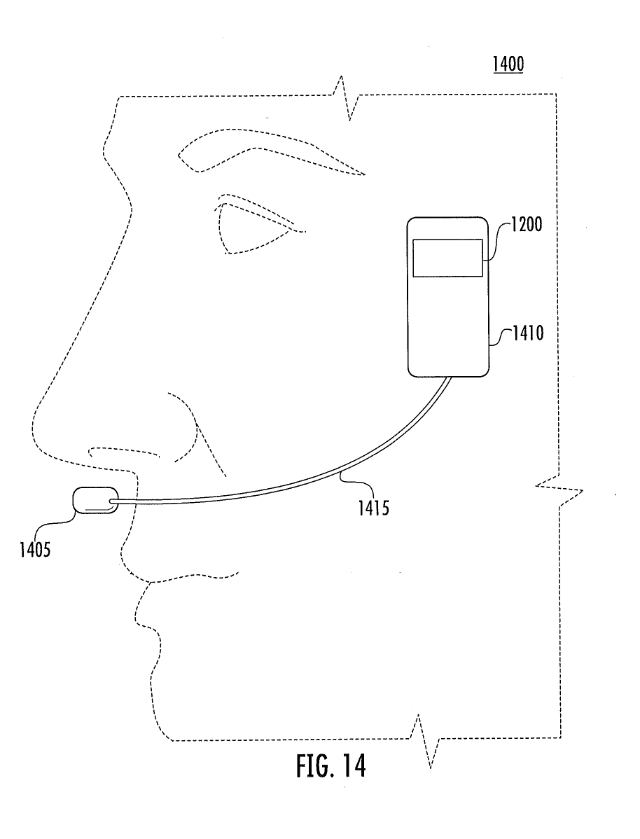

FIG. 14 is a schematic representation of a CO₂ detection system configured for operation in an open breathing environment in some embodiments according to the invention. According to FIG. 14, the color change material 100 along with the electronic emitter and a sensor as described herein can be located in an open environment. For example, adjacent to a subject's nose and/or mouth and not enclosed within, for example, the adapter shown in FIG. 8. According to FIG. 14, an open environment CO₂ detection system 1400 includes a sensor portion 1405 that can include the color change material 100 described herein. The sensor portion can also include a transmit/receive system which allows for the transmission of visible light from an emitter that is located remote from the sensor portion 1405. The transmit/receive system can also include a receiver that provides for the reflected visible light to be provided to an electronic sensor that is remote from the sensor portion 1405.

The CO₂ detection system 1400 also includes an electronic portion 1410 that can include the electronic emitter and electronic sensor in communication with the sensor portion 1405 via a transmission medium 1415 located therebetween. It will be understood that the electronics portion 1410 can also include a display such as that shown in FIG. 12 in some embodiments according to the invention. In operation, when the subject breathes in the open environment, sufficient CO₂ may be brought into contact with the color change material located in the sensor portion 1405 despite the fact that it is not enclosed within a breathing circuit as described herein. The remote electronics portion 1410 can be in communication with the sensor portion 1405 via the transmission media 1415 to provide the same determination of CO₂ levels included in the respiratory stream in the open environment.

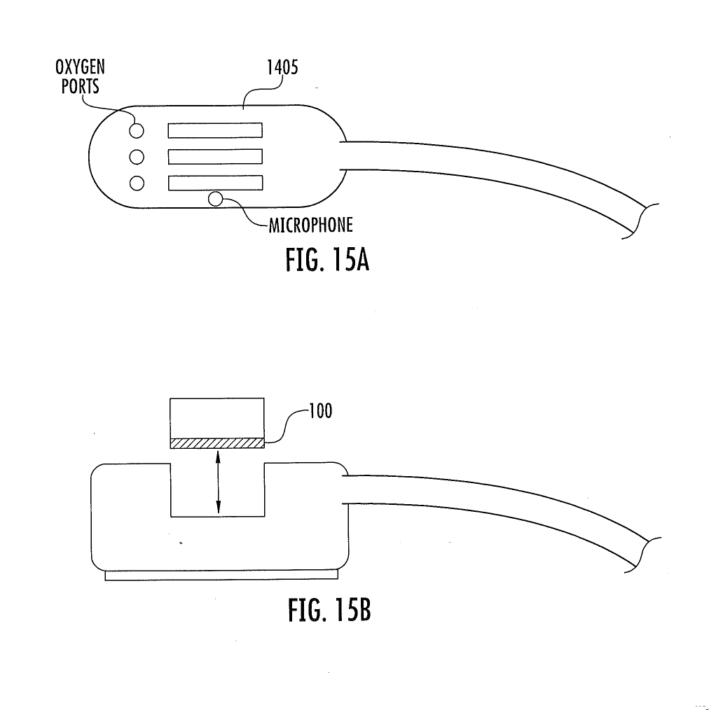

FIGS. 15A and 15B are different views of the CO₂ detection system 1400 shown in FIG. 14. According to FIG. 15A, the sensor portion 1405 can include ports that allow for the exhaled CO₂ to be in contact with the color change material located within. In addition, the sensor portion 1405 can include other features, such as, a microphone, oxygen ports, and other modalities and/or sensors. As shown in FIG. 1513, the color change material 100 may be included as part of an apparatus that is removably coupled to the sensor portion 1405. For example, the color change material 100 may be included as part of a cartridge that is inserted into the rear of the sensor portion 1405 so that the CO₂ detection system 1400 is not required to be removed from the subject for replacement of the color change material 100 such as when the buffer included in the color change indicator is depleted to the point where inaccurate CO₂ levels may be reported. Accordingly, other services to the subject, such as oxygen and other features may be uninterrupted while the CO₂ sensor color change material 100 is replaced.

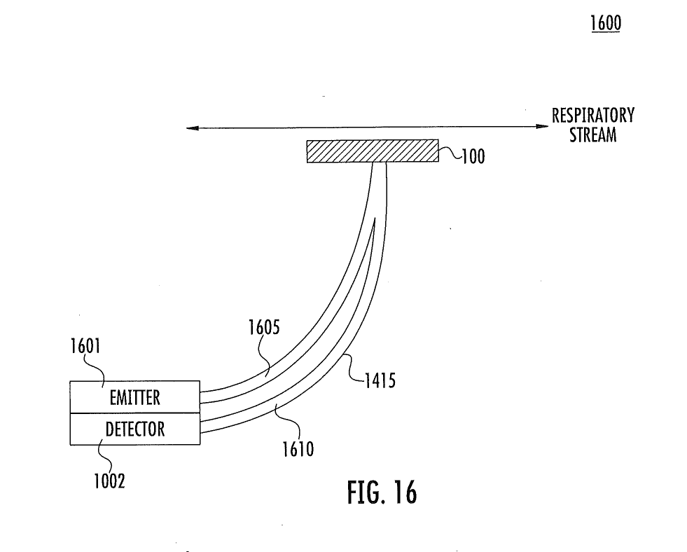

FIG. 16 is a schematic representation of an optical implementation of the CO₂ detection system 1400 shown in FIG. 14. According to FIG. 16, the color change material 100 can be located proximate to the respiratory stream as shown, for example, in FIG. 14 within the sensor portion 1405. The transmission medium 1415 can be provided by an optical cable that allows for the electronic emitter to provide the visible light to the color change material 100 via a first channel of the transmission medium, the first optical channel 1605 whereas the electronic sensor is provided with the reflected visible light via a second optical channel 1610. It will be understood that other types of transmission mediums may also be used.

It is also noted that circuitry designed for detecting CO₂ levels or other types of compounds may be small enough to be housed in a portable unit operating under battery power. The advantages of having a portable unit are numerous but may include availability in remote locations under in-the-field conditions. This may allow the detector to be provided to all EMT's, first responders, military units, police personnel and the like. Those of sufficient skill in the art appreciate that various types of batteries may be used to generate sufficient power to detect the presence of CO₂ as well as operate any type of display or data transmission.

Furthermore, the CO₂ detection system can be designed to be an all-in-one unit designed to display data or measurements at the actual point of measurement, which would be a display incorporated as part of the device that attaches to the endotrachael tube, ventilating mask, or source of the exhaled gases intended to be tested for the presence of CO₂. An alternative method would allow for remote monitoring of the collected data or measurements, via wireless connection to either a specifically designed, purpose built base unit which could either be hand held or bench top in nature, or via a specific application/app written to be used on a smart phone platform.

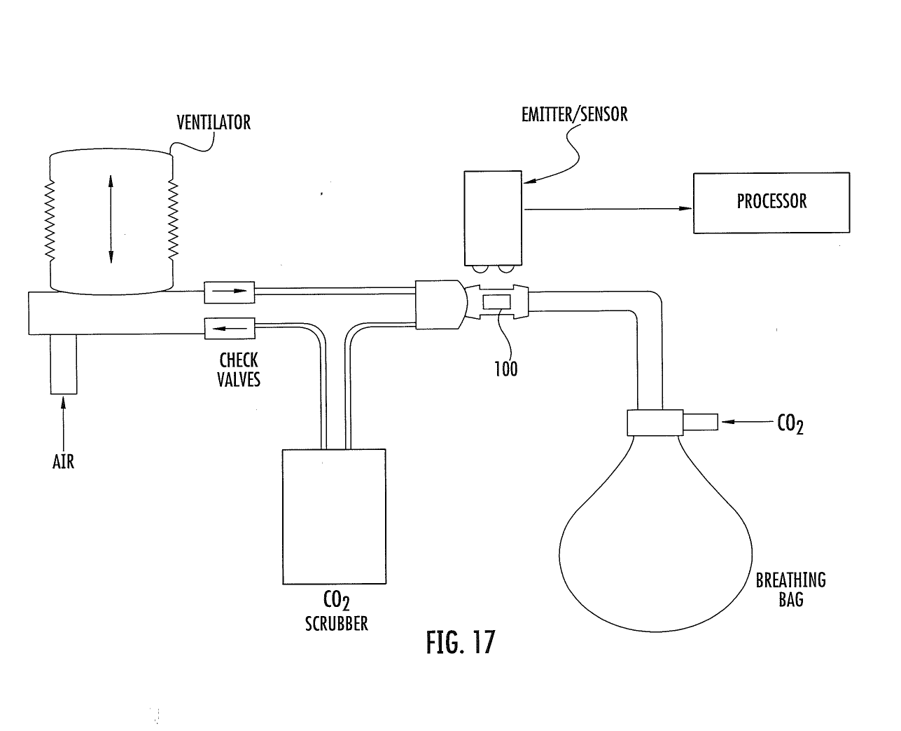

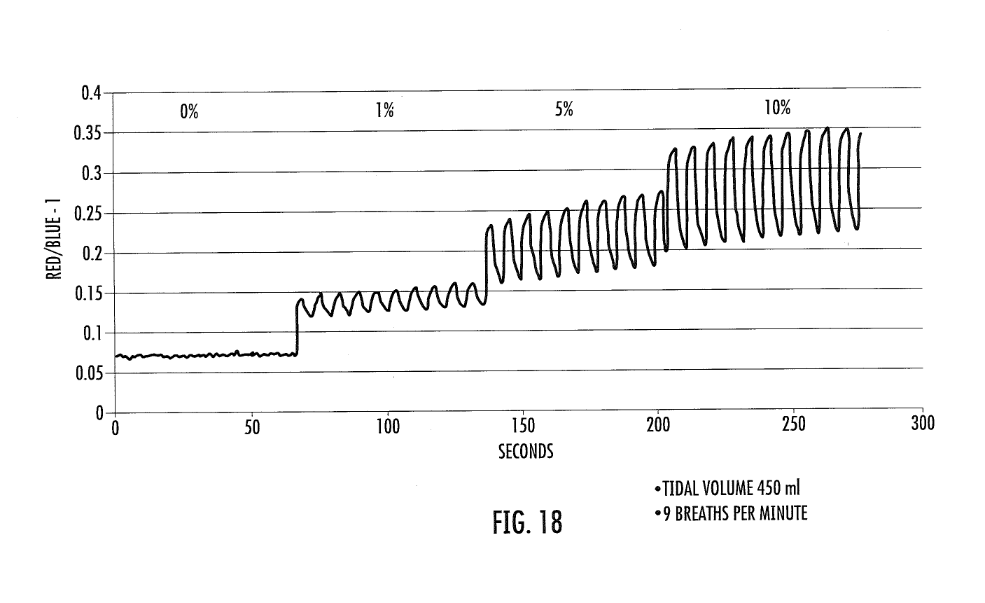

FIG. 17 is a schematic illustration of test setup for a CO₂ detection system in some embodiments according to the invention. FIG. 18 in a graph illustrating CO₂ information generated by the CO₂ detection system operating in the test setup of FIG. 17.

Carbon dioxide detector 1 was configured inside of a 21 mm adapter tube commonly used as a connector fitting in medical airway circuits. The color change material was mounted such that air flow within the tube was substantially parallel to the surface of the color change material, and the color change material was at a position approximately equatorial within the tube. The colorimetrically active surface of the color change material was illuminated from outside of the tube using a multicolor LED device containing a red, a green, and a blue LED in a surface mount package. A color sensing device was mounted adjacent the LED outside of the tube. The color sensing device was aimed at the surface of the color change material to intercept a portion of light reflected from its surface. The color sensing device was electronically configured to provide digital signals representative of the relative portions of red, green, and blue light in the reflected light.

Gas within the tube comprised a mixture of air and carbon dioxide, the relative proportions of which could be varied. The breathing circuit was connected to a respirator to simulate human breathing at 10 breaths per minute and a volume flow of 4 liters per minute. The gas circuit was configured to route gases through a “polysorb” carbon dioxide scrubber during the exhalation portion of the breathing cycle. This removed all CO₂ in the gas stream. CO₂ was mixed in a portion of the circuit to mimic production of CO₂ during an exhalation cycle. The “exhaled” breath was passed through the tube containing the color change material, and then routed to the scrubber. While breathing various mixtures of carbon dioxide that were intentionally varied from below normal physiological levels to above normal levels, data were recorded from the digital outputs of the color sensor device and plotted over time, as shown in FIG. 18. The plot showed that the average ratio of red color to blue color varied in proportion to the carbon dioxide content in the breath stream. The plot also showed that breath-to-breath differences in carbon dioxide could be recorded. Data was found to provide an accurate calibration of carbon dioxide content and respiratory rate.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present inventive subject matter. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

It will be understood that when an element or layer is referred to as being “on” another element or layer, the element or layer can be directly on another element or layer or intervening elements or layers may also be present. In contrast, when an element is referred to as being “directly on” another element or layer, there are no intervening elements or layers present. As used herein, the term “and/or” includes any and all combinations of one or more of the associated listed items.

Spatially relative terms, such as “below”, “beneath”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation, in addition to the orientation depicted in the figures. Throughout the specification, like reference numerals in the drawings denote like elements.

Embodiments of the inventive subject matter are described herein with reference to plan and perspective illustrations that are schematic illustrations of idealized embodiments of the inventive subject matter. As such, variations from the shapes of the illustrations as a result, for example, of manufacturing techniques and/or tolerances, are to be expected. Thus, the inventive subject matter should not be construed as limited to the particular shapes of objects illustrated herein, but should include deviations in shapes that result, for example, from manufacturing. Thus, the objects illustrated in the figures are schematic in nature and their shapes are not intended to illustrate the actual shape of a region of a device and are not intended to limit the scope of the inventive subject matter.

The terminology used herein is for the purpose of describing particular embodiments only and is not intended to be limiting of the present inventive subject matter. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present inventive subject matter belongs. It will be further understood that terms used herein should be interpreted as having a meaning that is consistent with their meaning in the context of this specification and the relevant art and will not be interpreted in an idealized or overly formal sense unless expressly so defined herein. The term “plurality” is used herein to refer to two or more of the referenced item.

It will be understood that, as used herein, the term light emitting device may include a light emitting diode, laser diode and/or other semiconductor device which includes one or more semiconductor layers, which may include silicon, silicon carbide, gallium nitride and/or other semiconductor materials, a substrate which may include sapphire, silicon, silicon carbide and/or other microelectronic substrates, and one or more contact layers which may include metal and/or other conductive layers.

In the drawings and specification, there have been disclosed typical preferred embodiments of the inventive subject matter and, although specific terms are employed, they are used in a generic and descriptive sense only and not for purposes of limitation, the scope of the inventive subject matter being set forth in the following claims.

1-15. (canceled)

16. A device comprising:
a visible light emitter circuit configured to provide emitted visible light into a breathing circuit;
a first visible light sensor circuit configured to receive a first portion of the emitted visible light; and
a processor circuit, coupled to the visible light emitter circuit and to the first visible light sensor circuit, the processor circuit configured to determine a CO2 level of a respiratory stream in the breathing circuit based on the first portion of the emitted visible light.

17. The device of claim 16 wherein the first visible light sensor circuit is configured to provide a reactive signal to the processor circuit as a color indication of the CO2 level based on the first portion of the emitted visible light.

18. The device of claim 16 wherein the visible light emitter circuit is located on a first side of the breathing circuit and the first visible light sensor circuit is located on a second side of the breathing circuit, opposite the first side.

19. The device of claim 16 wherein the visible light emitter circuit and the first visible light sensor circuit is located on a first side of the breathing circuit, the device further comprising:
a reflector on a second side of the breathing circuit, opposite the first side, positioned to reflect the emitted visible light from the visible light emitter circuit to the first visible light sensor circuit.

20. The device of claim 16 further comprising:
a color change material inside the breathing circuit overlying the first visible light sensor circuit, wherein the emitted visible light impinges a first surface of the color change material and the first portion of the emitted visible light exits a second surface of the color change material to impinge the first visible light sensor circuit.

21. The device of claim 16 wherein the processor circuit is configured to determine the CO2 level based on a comparison of at least two color components of the first portion of the emitted visible light.

22. The device of claim 21 wherein the at least two color components comprise red and green or red and blue.

23. The device of claim 20 wherein the visible light emitter circuit and the first visible light sensor circuit is remote from the respiratory stream, the device further comprising:
an optical transmission medium extending from the color change material to the visible light emitter circuit and to the first visible light sensor circuit.

24. The device of claim 16 wherein the breathing circuit comprises a conduit in which at least a portion of the respiratory stream is enclosed.

25. The device of claim 24 wherein the conduit is open to an ambient environment outside the respiratory stream.

26. The device of claim 16 wherein the breathing circuit comprises a conduit configured for direct coupling to an airway of a subject for which the CO2 level is determined.

27. The device of claim 24 wherein the processor circuit is configured to communicate the CO2 level to an electronic device that is remote from the device.

28. The device of claim 27 wherein the electronic device comprises a smartphone, tablet, or PDA.

29. A device comprising:
a visible light emitter circuit configured to provide emitted visible light into a respiratory stream;
a first visible light sensor circuit configured to receive a first portion of the emitted visible light; and
a processor circuit, coupled to the visible light emitter circuit and to the first visible light sensor circuit, the processor circuit configured to determine a CO2 level in the respiratory stream based on the first portion of the emitted visible light.

30. The device of claim 29 wherein the respiratory stream is unenclosed by a breathing circuit adapter.

31. The device of claim 29 further comprising:
a member configured for mounting of at least the first visible light sensor circuit thereon and configured for positioning within the respiratory stream.

32. The device of claim 31 wherein the member comprises an adjustable elongated member having a distal portion, outside the respiratory stream, configured for attachment to a subject for which the CO2 level is to be determined and a proximate portion for mounting the at least first visible light sensor circuit.

33. A composition for use in monitoring a respiratory stream comprising:
a color change material configured to change from a first color to a second color in response to an increase in CO₂ within the respiratory stream, where the first color includes more of a first component than a second component or more than a third component and the second color includes less of the first component than the second component or less than the third component.

34. A method of determining a CO₂ level of a respiratory stream, the method comprising:
electronically emitting visible light into the respiratory stream to provide emitted visible light onto at least a portion of a color change material in contact with the respiratory stream;
electronically sensing a first color generated by a reactive portion of color change material responsive to the emitted visible light; and
determining the CO2 level of the respiratory stream based on the first color.

35. The method of claim 34 wherein electronically sensing a first color comprises electronically sensing a first portion of the emitted visible light passing through the reactive portion of color change material as an indication of the first color, the method further comprising:
providing a reactive signal to a processor circuit as indication of the CO2 level based on the first portion of the emitting visible light.

36. The method of claim 35 wherein determining the CO2 level of the respiratory stream further comprises:
determining first color components of the first color; and
determining a first color ratio of the first color components to one another.

37. The method of claim 35 further comprising:
determining first color components of the first color;
determining a first color ratio of the first color components to one another; and
determining a respiration rate associated with the respiratory stream based on periodic determination of the first color ratio to provide a data set associated with at least one complete respiration cycle of the respiratory stream.

38. The method of claim 37 wherein determining the CO2 level of the respiratory stream further comprises:
determining the CO2 level based on a directly adjacent peak to peak values among the first color ratios in the data set and/or based on a minimum value of first color ratios in the data set.

39. The method of claim 38 wherein determining the CO2 level based on a directly adjacent peak to peak values among the first color ratios in the data set and/or based on a minimum value of first color ratios in the data set comprises:
determining the CO2 level based more on the directly adjacent peak to peak values than on the minimum value responsive to a first respiration rate; and
determining the CO2 level based more on the minimum value than on the directly adjacent peak to peak values responsive to a second respiration rate that is greater than the first respiration rate.
